(19) United States
(12) Patent Application Publication
(10) Pub. No.: US 2024/0352496 A1
(43) Pub. Date: Oct. 24, 2024

(54) IVT RNA MANUFACTURING PROCESS

(71) Applicant: GLAXOSMITHKLINE BIOLOGICALS SA, Rixensart (BE)

(72) Inventors: Justin Ross HALMAN, Rockville, MD (US); Nilesh VAIDYA, Rockville, MD (US)

(73) Assignee: GLAXOSMITHKLINE BIOLOGICALS SA, Rixensart (BE)

(21) Appl. No.: 18/687,892

(22) PCT Filed: Aug. 29, 2022
(86) PCT No.: PCT/IB2022/058089

§ 371 Date: Feb. 29, 2024

Related U.S. Application Data
(60) Provisional application No. 63/238,878, filed on Aug. 31, 2021.

(51) Int. Cl. C12P 19/34

(52) U.S. Cl. CPC C12P 19/34

(57) ABSTRACT

Improved methods for manufacturing a RNA by IVT and compositions for use therein are provided.

### FIELD OF THE INVENTION

The present invention relates to methods for manufacturing a RNA using a IVT reaction process, compositions for use therein, and related aspects thereof.

### BACKGROUND TO THE INVENTION

Recent advances in RNA production and formulation have made mRNA-based vaccine platforms possible. However, challenges still exist while manufacturing high quality mRNAs in large quantities, especially if the mRNAs are longer (e.g. self-amplyfiing mRNA) than conventional mRNA and/or have a high degree of secondary structure. The mRNAs maybe synthesized by in vitro transcription (IVT) of linearized DNA templates using an RNA polymerase that requires ribonucleotide triphosphates as RNA building blocks and a buffer system that includes DL-dithiothreitol (DTT) and magnesium ions as a cofactor to RNA polymerase. The IVT reaction may also include a ribonuclease inhibitor for inactivating RNase activity and pyrophosphatase for degrading accumulated pyrophosphate. Once RNA polymerase binds to its specific promoter sequence of the template, it may proceed along the DNA template synthesizing an RNA molecule and may be terminated at the 3′ end of the linear DNA molecule.

Cell free systems used to manufacture RNA using in vitro transcription require several reagents in a buffer system including DTT, magnesium ions, purified linear DNA template containing a promoter, ribonucleotide tri-phosphates (NTP), and an appropriate RNA polymerase. Commercially available reagents for manufacturing RNA include or recommend a buffer system for carrying out the reaction with specific concentrations of various components, such as 6-8 mM Mg²⁺ and 0.5-2 mM NTP. See the T7 RNA Polymerase kit from Promega or the T7 IVT kit from Thermo Invitrogen. Kern et al. (Kern J A, Davis R H. Application of a fed-batch system to produce RNA by in vitro transcription. Biotechnol Prog. 1999 March-April; 15(2):174-84. doi: 10.1021/bp990008g. PMID: 10194392/Kern, J. A. and Davis, R. H. (1997), Application of Solution Equilibrium Analysis to in vitro RNA Transcription. Biotechnol Progress, 13: 747-756. https://doi.org/10.1021/bp970094p) published an analysis of reaction buffer components, identifying Mg²⁺ and NTP concentration as two related parameters for a reaction buffer, arriving through linear regression analysis at a formula for determining recommended Mg²⁺ concentration as a function of total NTP concentration, specifically [Mg(OAc)₂]=1.55[Total NTP]+8.55. WO17/109161 discloses a wide range of Mg²⁺ and NTP concentrations, preferring an initial free Mg²⁺ concentration of about 24 mM.

Contemporary IVT protocols do not transcribe longer mRNAs efficiently due to their lengths as well as a high degree of secondary structures. There remains a need for new manufacturing approaches which enable improved yield of RNA without deleterious effects on the RNA integrity or subsequent manufacturing steps.

### SUMMARY OF THE INVENTION

Applicants provide compositions and methods for increasing RNA yield and quality during IVT manufacturing for both self-amplyfing RNA and non-replicating RNA.

In some embodiments of the invention, a method is provided for manufacturing a RNA by IVT having a step of combining components comprising a nucleic acid template; a RNA polymerase; and a suitable reaction buffer. A suitable buffer comprises Mg²⁺ at a concentration of 16-50 mM, such as 20-44 mM, 24-40 mM, 28-36 mM, 30-34 mM and NTPs at a total concentration of 20-52 mM, such as 24-46 mM, 30-42 mM, 34-38 mM. The method includes a step of manufacturing the RNA.

In some embodiments of the invention, a composition is provided comprising: (a) Mg²⁺ at a concentration of 16-50 mM, such as 20-44 mM, 24-40 mM, 28-36 mM, 30-34 mM; (b) NTPs at a total concentration of 20-52 mM, such as 24-46 mM, 30-42 mM, 34-38 mM.

In some embodiments of the invention, a method is provided for manufacturing a RNA by in vitro transcription (IVT), said method comprising the steps of (A) combining in a reaction vessel components comprising: (a) a nucleic acid template; (b) a RNA polymerase; and (c) a suitable reaction buffer, the buffer comprising (i) Mg²⁺ at a concentration of 32 mM; (ii) NTPs at a total concentration of 36 mM; (B) manufacturing the RNA; and (C) capping the 5′ end of the RNA.

In some embodiments of the invention, a method is provided for manufacturing a pharmaceutically acceptable RNA by in vitro transcription (IVT), said method comprising the steps of (A) combining in a reaction vessel components comprising: (a) a nucleic acid template; (b) a RNA polymerase; and (c) a suitable reaction buffer, the buffer comprising (i) Mg²⁺ at a total concentration of 16-50 mM, such as 20-44 mM, 24-40 mM, 28-36 mM, 30-34 mM; (ii) NTPs at a concentration of 20-52 mM, such as 24-46 mM, 30-42 mM, 34-38 mM; (B) manufacturing the RNA; (C) purifying and packaging the RNA.

The conditions for manufacturing mRNA via IVT may change depending on the size of the mRNA (self-replicating vs non-replicating). Therefore, concentrations, temperature, etc. may differ depending on the size and/or type of mRNA.

In alternative embodiments of the invention, a method is provided for manufacturing a stable and translatable mRNA by in vitro transcription (IVT), said method comprising the steps of (A) combining in a reaction vessel components comprising: (a) a nucleic acid template; (b) a RNA polymerase; and (c) a suitable reaction buffer, the buffer comprising (i) Mg²⁺ at a total concentration of 5-48 mM, such as 6-30 mM, 7-25 mM, 8-20 mM; (ii) NTPs at a concentration of 8-40 mM, such as 12-28 mM; (B) manufacturing the RNA.

In some embodiments of the invention, a composition is provided comprising: (a) Mg²⁺ at a concentration of 5-48 mM, such as 6-30 mM, 7-25 mM, 8-20 mM; (b) NTPs at a total concentration of 8-40 mM, such as 12-28 mM.

In some embodiments of the invention, a method is provided for manufacturing a stable and translatable mRNA by in vitro transcription (IVT), said method comprising the steps of (A) combining in a reaction vessel components comprising: (a) a nucleic acid template; (b) a RNA polymerase; and (c) a suitable reaction buffer, the buffer comprising (i) Mg²⁺ at a total concentration of 5-48 mM, such as 6-30 mM, 7-25 mM, 8-20 mM; (ii) NTPs at a concentration of 8-40 mM, such as 12-28 mM; (B) manufacturing the RNA.

In some embodiments of the invention, a method is provided for manufacturing a non-replicating RNA by in vitro transcription (IVT), said method comprising the steps of (A) combining in a reaction vessel components comprising: (a) a nucleic acid template; (b) a RNA polymerase; and (c) a suitable reaction buffer, the buffer comprising (i) Mg²⁺ at a concentration of 16 mM; (ii) NTPs at a total concentration of 20 mM; (B) manufacturing the RNA; and (C) capping the 5′ end of the RNA.

In some embodiments of the invention, a method is provided for manufacturing a pharmaceutically acceptable RNA by in vitro transcription (IVT), said method comprising the steps of (A) combining in a reaction vessel components comprising: (a) a nucleic acid template; (b) a RNA polymerase; and (c) a suitable reaction buffer, the buffer comprising (i) Mg²⁺ at a total concentration of 5-48 mM, such as 6-30 mM, 7-25 mM, 8-20 mM; (ii) NTPs at a concentration of 8-40 mM, such as 12-28 mM; (B) manufacturing the RNA; (C) purifying and packaging the RNA.

In some embodiments of the invention, a method is provided for manufacturing a pharmaceutically acceptable mRNA by in vitro transcription (IVT), said method comprising the steps of (A) combining in a reaction vessel components comprising: (a) a nucleic acid template; (b) a RNA polymerase; and (c) a suitable reaction buffer, the buffer comprising (i) Mg²⁺ at a total concentration of 5-48 mM, such as 6-30 mM, 7-25 mM, 8-20 mM; (ii) NTPs at a concentration of 8-40 mM, such as 12-28 mM; (B) manufacturing the RNA; (C) capping the 5′ end of the RNA; (D) purifying and packaging the capped RNA.

### DESCRIPTION OF DRAWINGS/FIGURES

FIG. 1 shows significant components and interactions. The components and interactions with a logworth>2 or ϵ-value <0.005 is considered significant.

FIG. 2 shows the coefficient estimates of each factor and secondary interactions for the yield of RNA.

FIG. 3 shows the coefficient estimates of each factor and secondary interactions for the percentage of full-length RNA.

FIG. 4 shows the effects of varying each factor on RNA yield and RNA quality is plotted next to each other to understand the relationship between each factor in order to produce high RNA yield and quality.

FIG. 5 shows the significant components and interactions with a logworth >2 or P-value <0.005.

FIG. 6 shows the high-resolution interaction between four factors at different levels on the IVT yield and the RNA quality (percentage of full-length RNA).

FIG. 7 shows the effects of varying NTP, Magnesium and IVT temperature on the RNA yield and the RNA quality plotted next to each other to understand the relationship between each factor in order to produce high RNA yield and high-quality RNA.

FIG. 8 shows the selection of conditions for IVT varying the NTP:Mg²⁺ ratios

### DETAILED DESCRIPTION OF THE INVENTION

### General

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. The term “plurality” refers to two or more. Additionally, numerical limitations given with respect to concentrations or levels of a substance, such as solution component concentrations or ratios thereof, and reaction conditions such as temperatures, pressures and cycle times are intended to be approximate.

“Comprise” (“comprising” or “comprises”) as used herein is open-ended and means “including, but not limited to.” “Having” is used herein as a synonym of comprising. It is understood that wherever embodiments are described herein with the language “comprising,” such embodiments encompass those described in terms of “consisting of” and/or “consisting essentially of”.

“About” or “approximately” mean roughly, around, or in the regions of. The terms “about” or “approximately” further mean within an acceptable contextual error range for the particular value as determined by one of ordinary skill in the art, which will depend in part on how the value is measured, i.e. the limitations of the measurement system or the degree of precision required for a particular purpose. When the terms “about” or “approximately” are used in conjunction with a numerical range, it modifies that range by extending the boundaries above and below the numerical values set forth.

The term “and/or” as used in a phrase such as “A and/or B” is intended to include “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless specifically stated, a process comprising a step of mixing two or more components does not require any specific order of mixing. Components can be mixed in any order. Where there are three components then two components can be combined with each other, and then the combination may be combined with the third component, etc. Similarly, while steps of a method may be numbered (such as (1), (2), (3), etc. or (i), (ii), (iii)), the numbering of the steps does not itself mean that the steps must be performed in that order (i.e., step 1 then step 2 then step 3, etc.). In certain embodiments, the word “then” is used to specify the order of a method's steps.

“Essentially the same” herein means a high degree of similarity between at least two molecules (including structure or function) or numeric values such that one of skill in the art would consider the difference to be immaterial, negligible, and/or statistically insignificant. “Essentially the same” herein encompasses “the same.”

“Subject” refers to an animal, in particular a mammal such as a primate (e.g. human).

“Essentially free,” as in “essentially free from” or “essentially free of,” means comprising less than a detectable level of a referenced material or comprising only unavoidable levels of a referenced material (trace amounts).

The word “substantially” does not exclude “completely” e.g. a composition which is “substantially free” from Y may be completely free from Y. “Substantially pure” refers to material which is at least 50% pure (i.e., free from contaminants), at least 90% pure, at least 95% pure, at least 98% pure, or at least 99% pure.

“Buffer system” herein comprises a buffer e.g. a phosphate buffer, a Tris buffer, a borate buffer, a succinate buffer, a histidine buffer, or a citrate buffer.

“Capping” is a process that utilizes the enzymes RNA triphosphatase, guanylyltransferase, and methyltransferase. Through a series of steps, the cap is added to the first nucleotide's 5′ hydroxyl group of the growing mRNA strand while transcription is still occurring. RNA 5′ triphosphatase hydrolyzes the 5′ triphosphate group to make diphospate-RNA. The addition of GMP by guanylyltransferase produces the guanosine cap. RNA methyltransferase transfers a methyl group to the guanosine cap to yield 7-methylguanosine cap that is attached to the 5′ end of the transcript. These three enzymes, collectively called the capping enzymes, are able to catalyze their respective reactions when attached to RNA polymerase II, an enzyme necessary for the transcription of DNA into pre-mRNA. When this complex of RNA polymerase II and the capping enzymes is achieved, the capping enzymes are able to add the cap to the mRNA while it is produced by RNA polymerase II.

“Clean-cap®” herein means a co-transcriptional 5′ capping solution that generates a natural Cap 1 structure. Proper mRNA capping is critical to the production of the most biologically active and least immunogenic mRNA.

“mRNA/messenger-RNA” herein means a single-stranded RNA molecule that is complementary to one of the DNA strands of a gene. The mRNA is an RNA version of the gene that leaves the cell nucleus and moves to the cytoplasm where proteins are made. During protein synthesis, an organelle called a ribosome moves along the mRNA, reads its base sequence, and uses the genetic code to translate each three-base triplet, or codon, into its corresponding amino acid.

The term mRNA may include conventional mRNA or mRNA analogs, such as those containing modified backbones or modified bases. mRNA may comprise a sequences which encodes at least one antigen.

“NTP” herein means nucleotide triphosphates that a RNA polymerase can incorporate into a RNA molecule and may include naturally-occurring and non-naturally occurring nucleotide triphosphates. A nucleoside triphosphate is a molecule containing a nitrogenous base bound to a 5-carbon sugar (either ribose or deoxyribose), with three phosphate groups bound to the sugar. They are the molecular precursors of both DNA and RNA, which are chains of nucleotides made through the processes of DNA replication and transcription. Examples of NTPs include but are not limited to: ATP, GTP, m⁵UDP, UTP, CTP, ITP, XTP, dATP, dGTP, dTTP, dUTP, dCTP, dITP, and dXTP.

“NTP-NTP interactions” herein means the interactions between individual NTPs as a result of possible hydrogen bonding.

“Nucleic acid” herein means a polymeric chain of nucleotides of any length, which contain deoxyribonucleotides, ribonucleotides, and/or their analogs. It includes DNA, RNA and DNA/RNA hybrids. It also includes DNA or RNA analogs, such as those containing modified backbones (e.g. peptide nucleic acids (PNAs) or phosphorothioates) or modified bases. Thus the nucleic acid of the disclosure includes mRNA, DNA, cDNA, recombinant nucleic acids, branched nucleic acids, plasmids, vectors, etc. Where the nucleic acid takes the form of RNA, it may or may not have a 5′ cap. RNA may be a small, medium, or large RNA. The number of nucleotides per strand of a small RNA is from 10-30 (e.g. siRNAs). A medium RNA contains between 30-2000 nucleotides per strand (e.g. non-self-replicating mRNAs). A large RNA contains at least 2,000 nucleotides per strand e.g. at least 2,500, at least 3,000, at least 4,000, at least 5,000, at least 6,000, at least 7,000, at least 8,000, at least 9,000, at least 10,000 at least 15,000, at least 20,000, or at least 25,000 nucleotides per strand (e.g. self-replicating RNAs as described below). The molecular mass of a single-stranded RNA molecule in g/mol (or Dalton) can be approximated using the formula: molecular mass=(number of RNA nucleotides)×340 g/mol. An RNA (particularly a self-replicating RNA) can include, in addition to any 5′ cap structure, one or more nucleotides having a modified nucleobase. For instance, a RNA can include one or more modified pyrimidine nucleobases, such as pseudouridine and/or 5 methylcytosine residues. In some embodiments, however, the RNA includes no modified nucleobases, and may include no modified nucleotides i.e. all of the nucleotides in the RNA are standard A, C, G and U ribonucleotides (except for any 5′ cap structure, which may include a 7′ methylguanosine). In other embodiments, the RNA may include a 5′ cap comprising a 7′ methylguanosine, and the first 1, 2 or 3 5′ ribonucleotides may be methylated at the 2′ position of the ribose.

Nucleic acids can be in recombinant form, i. e. a form which does not occur in nature. For example, the nucleic acid may comprise one or more heterologous nucleic acid sequences (e.g. a sequence encoding another antigen and/or a control sequence such as a promoter or an internal ribosome entry site). The nucleic acid may be part of a vector i.e. part of a nucleic acid designed for transduction/transfection of one or more cell types. Vectors may be, for example, “expression vectors” which are designed for expression of a nucleotide sequence in a host cell, or “viral vectors” which are designed to result in the production of a recombinant virus or virus-like particle.

Alternatively, or in addition, the sequence or chemical structure of the nucleic acid may be modified compared to a naturally-occurring sequence. The sequence of the nucleic acid molecule may be modified, e.g. to increase the efficacy of expression or replication of the nucleic acid, or to provide additional stability or resistance to degradation.

In some embodiments, the nucleic acid may be codon optimized for expression in human cells. By “codon optimized” is intended modification with respect to codon usage which may increase translation efficacy and/or half-life of the nucleic acid. A poly A tail (e.g., of about 30 adenosine residues or more) may be attached to the 3′ end of the RNA to increase its half-life. The 5′ end of the RNA may be capped with a modified ribonucleotide with the structure m7G (5) ppp (5) N (cap 0 structure) or a derivative thereof, which can be incorporated during RNA synthesis or can be enzymatically engineered after RNA transcription (e.g., by using Vaccinia Virus Capping Enzyme (VCE) consisting of mRNA triphosphatase, guanylyl-transferase and guanine-7-methytransferase, which catalyzes the construction of N7-monomethylated cap 0 structures). Cap 0 structure plays an important role in maintaining the stability and translational efficacy of the RNA molecule. The 5′ cap of the RNA molecule may be further modified by a 2 ′-O-Methyltransferase which results in the generation of a cap 1 structure (m7Gppp [m2 ′-O] N), which may further increase translation efficacy.

The nucleic acids may comprise one or more nucleotide analogs or modified nucleotides. As used herein, “nucleotide analog” or “modified nucleotide” refers to a nucleotide that contains one or more chemical modifications (e.g., substitutions) in or on the nitrogenous base (nucleobase) of the nucleoside (e.g. cytosine (C), thymine (T) or uracil (U), adenine (A) or guanine (G)). A nucleotide analog can contain further chemical modifications in or on the sugar moiety of the nucleoside (e.g., ribose, deoxyribose, modified ribose, modified deoxyribose, six-membered sugar analog, or open-chain sugar analog), or the phosphate. Many modified nucleosides and modified nucleotides are commercially available.

Modified nucleobases which can be incorporated into modified nucleosides and nucleotides and be present in the RNA molecules include: m5C (5-methylcytidine), m5U (5-methyluridine), m6A (N6-methyladenosine), s2U (2-thiouridine), Um (2′-O-methyluridine), mIA (1-methyladenosine); m2A (2-methyladenosine); Am (2-1-O-methyladenosine); ms2m6A (2-methylthio-N6-methyladenosine); i6A (N6-isopentenyladenosine); ms2i6A (2-methylthio-N6isopentenyladenosine); io6A (N6-(cis-hydroxyisopentenyl)adenosine); ms2io6A (2-methylthio-N6-(cis-hydroxyisopentenyl) adenosine); g6A (N6-glycinylcarbamoyladenosine); t6A (N6-threonyl carbamoyladenosine); ms2t6A (2-methylthio-N6-threonyl carbamoyladenosine); m6t6A (N6-methyl-N6-threonylcarbamoyladenosine); hn6A(N6-hydroxynorvalylcarbamoyl adenosine); ms2hn6A (2-methylthio-N6-hydroxynorvalyl carbamoyladenosine); Ar(p) (2′-0-ribosyladenosine (phosphate)); I (inosine); mil (1-methylinosine); m′Im (I 2′-0-dimethylinosine); m3C (3-methylcytidine); Cm (2T-0-methylcytidine); s2C (2-thiocytidine); ac4C (N4-acetylcytidine); 5C (5-fonnylcytidine); m5Cm (5,2-0-dimethylcytidine); ac4Cm (N4acetyl2TOmethylcytidine); k2C (lysidine); mIG (1-methylguanosine); m2G (N2-methylguanosine); m7G (7-methylguanosine); Gm (2′-0-methylguanosine); m22G (N2,N2-dimethylguanosine); m2Gm (N2,2′-0-dimethylguanosine); m22Gm (N2,N2,2′-0-trimethylguanosine); Gr(p) (2′-0-ribosylguanosine (phosphate)); yW (wybutosine); o2yW (peroxywybutosine); OHyW (hydroxywybutosine); OHyW* (undermodified hydroxywybutosine); imG (wyosine); mimG (methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G* (archaeosine); D (dihydrouridine); m5Um (5,2′-0-dimethyluridine); s4U (4-thiouridine); m5s2U (5-methyl-2-thiouridine); s2Um (2-thio-2′-0-methyluridine); acp3U (3-(3-amino-3-carboxypropyl)uridine); ho5U (5-hydroxyuridine); mo5U (5-methoxyuridine); cmo5U (uridine 5-oxyacetic acid); mcmo5U (uridine 5-oxyacetic acid methyl ester); chm5U (5-(carboxyhydroxymethyl)uridine)); mchm5U (5-(carboxyhydroxymethyl)uridine methyl ester); mcm5U (5-methoxycarbonyl methyluridine); mcm5Um (S-methoxycarbonylmethyl-2-0-methyluridine); mcm5s2U (5-methoxycarbonylmethyl-2-thiouridine); nm5s2U (5-aminomethyl-2-thiouridine); mnm5U (5-methylaminomethyluridine); mnm5s2U (5-methylaminomethyl-2-thiouridine); mnm5se2U (5-methylaminomethyl-2-selenouridine); ncm5U (5-carbamoylmethyl uridine); ncm5Um (5-carbamoylmethyl-2′-0-methyluridine); cmnm5U (5-carboxymethylaminomethyluridine); cnmm5Um (5-carboxymethy 1 aminomethyl-2-L-Omethyl uridine); cmnm5s2U (5-carboxymethylaminomethyl-2-thiouridine); m62A (N6,N6-dimethyladenosine); Tm (2′-0-methylinosine); m4C (N4-methylcytidine); m4Cm (N4,2-0-dimethylcytidine); hm5C (5-hydroxymethylcytidine); m3U (3-methyluridine); cm5U (5-carboxymethyluridine); m6Am (N6,T-0-dimethyladenosine); rn62Am (N6,N6,0-2-trimethyladenosine); m2′7G (N2,7-dimethylguanosine); m2′2′7G (N2,N2,7-trimethylguanosine); m3Um (3,2T-0-dimethyluridine); m5D (5-methyldihydrouridine); f5Cm (5-formyl-2′-0-methylcytidine); mlGm (I 2′-0-dimethylguanosine); m′Am (1,2-0-dimethyl adenosine) irinomethyluridine); tm5s2U (S-taurinomethyl-2-thiouridine)); iniG-14 (4-demethyl guanosine); imG2 (isoguanosine); ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(Ci-Ce)-alkyluracil, 5-methyluracil, 5-(C2-C6)-alkenyluracil, 5-(C2-Ce)-alkynyluracil, 5-(hydroxymethyl)uracil, 5-chlorouracil, 5-fluorouracil, 5-bromouracil, 5-hydroxycytosine, 5-(C1-C6)-alkylcytosine, 5-methylcytosine, 5-(C2-C6)-alkenylcytosine, 5-(C2-C6)-alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, hydrogen (abasic residue), m5C, m5U, m6A, s2U, W, or 2′-0-methyl-U. Many of these modified nucleobases and their corresponding ribonucleosides are available from commercial suppliers.

“Nucleic acid template” herein comprises DNA molecules, DNA protein, or RNA, (Li Zhou, Jinsong Ren, Xiaogang Qu, Nucleic acid-templated functional nanocomposites for biomedical applications, Materials Today, Volume 20, Issue 4, 2017, Pages 179-190, ISSN 1369-7021).

“One factor at a time (OFAT)” herein means a method of designing experiments involving the testing of factors, or causes, one at a time instead of multiple factors simultaneously.

“RNA” is the usual abbreviation for ribonucleic acid. It is a nucleic acid molecule, i.e. a polymer consisting of nucleotide monomers. These nucleotides are usually adenosine-monophosphate (AMP), uridine-monophosphate (UMP), guanosine-monophosphate (GMP) and cytidine-monophosphate (CMP) monomers or analogs thereof, which are connected to each other along a so-called backbone. The backbone is formed by phosphodiester bonds between the sugar, i.e. ribose, of a first and a phosphate moiety of a second, adjacent monomer. The specific order of the monomers, i.e. the order of the bases linked to the sugar/phosphate-backbone, is called the RNA sequence. Usually RNA may be obtainable by transcription of a DNA sequence, e.g., inside a cell.

“RNA quality” herein means the percentage of full-length RNA consisting of entire sequence encoded in DNA template to the total RNA (full-length and truncated RNA species) produced during IVT reaction.

“RNA polymerase” is an enzyme that is responsible for copying a DNA sequence into an mRNA sequence, during the process of transcription. As complex molecule composed of protein subunits, RNA polymerase controls the process of transcription, during which the information stored in a molecule of DNA is copied into a new molecule of messenger RNA.

“Self-amplifying” or “self-replicating” (used interchangeably) herein means, in relation to RNA or mRNA, the ability to replicate itself. The self-amplifying RNA molecules of the invention comprise mRNA encoding one or more antigens. The RNA can be produced in vitro by enzymatic transcription, thereby avoiding manufacturing issues associated with cell culture production of vaccines. After immunization with a self-amplifying RNA molecule of the invention, replication and amplification of the RNA molecule occur in the cytoplasm of the transfected cell and the nucleic acid is not integrated into the genome. As the RNA does not integrate into the genome and transform the target cell, self-amplifying RNA vaccines do not pose the safety hurdles faced by recombinant DNA vaccines.

Self-amplifying RNAs contain the basic elements of mRNA, i.e., a cap, 5′UTR, 3′UTR and a poly(A) tail. They may comprise a large open reading frame (ORF) that encodes non-structural viral genes and one or more subgenomic promoter. The nonstructural genes, which include a polymerase, form intracellular RNA replication factories and transcribe the subgenomic RNA at high levels. This mRNA encoding the vaccine antigen(s) is amplified in the cell, resulting in high levels of mRNA and antigen expression.

Self-replicating RNA molecules can have various lengths, but they may be 5000-25000 nucleotides long. Self-replicating RNA molecules may be single-stranded. Single-stranded RNAs can generally initiate an adjuvant effect by binding to TLR7, TLR8, RNA helicases and/or protein kinase R (PKR). RNA delivered in double-stranded form (dsRNA) can bind to TLR3, and this receptor can also be triggered by dsRNA which is formed either during replication of a single-stranded RNA or within the secondary structure of a single-stranded RNA.

The self-replicating RNA can be prepared by in vitro transcription (IVT). IVT can use a template created and propagated in plasmid form in bacteria, or created synthetically (for example by gene synthesis and/or polymerase chain-reaction (PCR) engineering methods). For instance, a DNA-dependent RNA polymerase (such as the bacteriophage T7, T3 or SP6 RNA polymerases) can be used to transcribe the self-replicating RNA from a DNA template. Appropriate capping and poly-A addition reactions can be used as required (although the replicon's poly-A is usually encoded within the DNA template). These RNA polymerases can have stringent requirements for the transcribed 5′ nucleotide(s) and in some embodiments these requirements must be matched with the requirements of the encoded replicase, to ensure that the IVT-transcribed RNA can function efficiently as a substrate for its self-encoded replicase.

A self-replicating RNA can include (in addition to any 5′ cap structure) one or more nucleotides having a modified nucleobase. A RNA used with the invention ideally includes only phosphodiester linkages between nucleosides, but in some embodiments it can contain phosphoramidate, phosphorothioate, and/or methylphosphonate linkages.

The word “stability” refers to the extent to which an RNA molecule retains its structural integrity and resists degradation during physical or chemical manipulations. For example, if RNA stability remains unchanged after purification, then the level of structural integrity has not changed, for example measured by analysing the average RNA size or the RNA size distribution.

“Stable and translatable mRNA” herein means mRNA that is full-length and has all the essential components (cap, tail, UTRs) to be translatable.

A “suitable reaction buffer” is a buffering system with all the necessary components for RNA polymerase (e.g. T7, 73 or SP6) to be functional. For example, A suitable buffer comprises Mg²⁺ at a concentration of 16-50 mM, such as 20-44 mM, 24-40 mM, 28-36 mM, 30-34 and NTPs at a total concentration of 20-52 mM, such as 24-46 mM, 30-42, 34-38 mM. An example of an alternate suitable buffers, comprises Mg²⁺ at a concentration of 5-48 mM, such as 6-30 mM, 7-25 mM, 8-20 mM

“T7 RNA polymerase” is an RNA polymerase from the T7 bacteriophage that catalyzes the formation of RNA from DNA in the 5->3′ direction.

“Total NTPs” herein refers to all NTPs in a composition, such as a solution. Total NTPs may be described in various units, for example, as molar amounts present in a solution.

In vitro transcription (IVT) is a procedure that allows for template-directed synthesis of RNA molecules of any sequence from short oligonucleotides to those of several kilobases in μg to mg quantities. It is based on the engineering of a template that includes a bacteriophage promoter sequence (e.g. from the T7 coliphage) upstream of the sequence of interest followed by transcription using the corresponding RNA polymerase. The basic strategy is to place the sequence of interest downstream from the promoter (ideally T7). The promoter covers the sequence ranging from −17 to +6 with +1 being the first nucleotide of the transcribed region. The template for transcription can be (1) a plasmid that typically has the promoter for in vitro transcription immediately upstream from a polylinker for cloning the sequence to be transcribed, (2) a PCR product that has the T7 promoter as part of the 5′-oligonucleotide used in the PCR reaction, and (3) two annealed oligonucleotides that carry the T7 promoter sequence and the template to be transcribed (in this case, only the T7 promoter part of the template needs to be double-stranded). An alternative strategy consists in cloning a DNA fragment including a promoter (ideally T7) immediately 5′ of the sequence to be transcribed in order to avoid the presence of nucleotides derived from multiple cloning sites (MCS) in the transcript. Cloned templates are used for long transcripts (>100 nt) and annealed oligo's for very short transcripts. When large amounts of RNA are needed, it is better to use a cloned template in order to generate enough template using simple and economical techniques based on bacterial culture and plasmid extraction. When small amounts are needed, PCR-products are probably the most convenient due to the flexibility in design of the template and the ease of its production. (Beckert B, Masquida B. Synthesis of RNA by in vitro transcription. Methods Mol Biol. 2011; 703:29-41. doi: 10.1007/978-1-59745-248-9_3. PMID: 21125481.)

General steps in synthesis of RNA molecules via plasmid templates may include but are not limited to the following steps: digestion of the plasmid DNA wherein a restriction enzyme cleaves downstream of the promoter (e.g. T7), adding proteinase and incubating to remove the restriction enzyme, extraction, precipitation, and resuspension. General steps in synthesis of RNA molecules via PCR templates may include but are not limited to the following steps: designing the oligios for PCR-amplification, making a standard PCR reaction, and purifying the product. (Beckert B, Masquida B. Synthesis of RNA by in vitro transcription. Methods Mol Biol. 2011; 703:29-41. doi: 10.1007/978-1-59745-248-9_3. PMID: 21125481). Changing reaction conditions can have disparate impacts on various characteristics of the resulting RNA. Example 4 shows that you may get high yield but low quality, or high quality (full-length RNA) but low yield, depending on which parameters are varied and it is not predicatable which parameters lead to which outcome.

IVT can use a template created and propagated in plasmid form in bacteria, or created synthetically, for example by gene synthesis and/or polymerase chain-reaction (PCR) engineering methods. For example, a DNA-dependent RNA polymerase, such as the bacteriophage T7, T3 or SP6 RNA polymerases, can be used to transcribe the self-amplifying RNA from a DNA template. Appropriate capping and poly-A addition reactions can be used as required (although the replicon's poly-A is usually encoded within the DNA template). These RNA polymerases can have stringent requirements for the transcribed 5′ nucleotide(s) and in some embodiments these requirements must be matched with the requirements of the encoded replicase, to ensure that the IVT-transcribed RNA can function efficiently as a substrate for its self-encoded replicase.

Individual factors such as IVT temperature, template DNA concentration, RNA polymerase concentration, magnesium concentration, nucleotide concentration, DL-dithiothreitol concentration, buffer concentration, and their interactions have an impact on IVT yield and RNA quality. The present inventors found total NTPs concentrations ranging from 20-52 mM, Mg²⁺ concentrations ranging from 16-50 mM, and temperature at 20-40° C. resulted in improved yields and quality of RNA.

Plasmid DNA encoding alphavirus replicons (named: pT7-mVEEV-FL.RSVF or A317; pT7-mVEEV-SEAP or A306; pSP6-VCR-GFP or A50) serve as a template for synthesis of RNA in vitro. A bacteriophage (T7 or SP6) promoter upstream of the alphavirus cDNA facilitates the synthesis of the replicon RNA in vitro. Following linearization of the plasmid DNA, (with a suitable restriction endonuclease) run-off transcripts are synthesized in vitro using T7 or SP6 bacteriophage derived DNA-dependent RNA polymerase. Transcription is performed for 2 hours at 37° C. in the presence of 7.5 mM (T7 RNA polymerase) or 5 mM (SP6 RNA polymerase) of each of the nucleoside triphosphates (ATP, CTP, GTP and UTP). Following transcription the template DNA is digested with TURBO DNase (Ambion). RNA is precipitated with a salt (e.g. LiCl) and reconstituted in nuclease-free water. Uncapped RNA is capped post-transcriptionally with Vaccinia Capping Enzyme (VCE) using the ScriptCap m7G Capping System (Epicentre Biotechnologies) as outlined in the user manual. Post-transcriptionally capped RNA is precipitated with a salt (e.g. LiCl) and reconstituted in nuclease-free water. The concentration of the RNA samples is determined by measuring OD260 nm-Integrity of the in vitro transcripts was confirmed by denaturing agarose gel electrophoresis.

### Self-Amplifying RNA

In some embodiments of the invention, a method is provided for manufacturing a RNA by IVT comprising a step of combining components comprising a nucleic acid template; a RNA polymerase; and a reaction buffer, wherein the reaction buffer comprises Mg²⁺ at a concentration of 16-50 mM, such as 20-44 mM, 24-40 mM, 28-36 mM, 30-34 mM and NTPs at a total concentration of 20-52 mM, such as 24-46 mM, 30-42 mM, 34-38 mM, and a step of manufacturing the RNA.

In some embodiments of the invention, a method is provided for manufacturing a RNA by IVT comprising (i) a step of combining reaction components comprising a nucleic acid template; a RNA polymerase; and a reaction buffer, wherein the reaction buffer comprises Mg²⁺ at a concentration of 16-50 mM, such as 20-44 mM, 24-40 mM, 28-36 mM, 30-34 mM and NTPs at a total concentration of 20-52 mM, such as 24-46 mM, 30-42 mM, 34-38 mM, and (ii) a step of incubating the reaction components to produce the RNA.

In certain embodiments, the step of incubating the reaction components is carried out at 20-40° C., such as 25-35° C., 27-33° C., 28-32° C., 29-31° C. in certain embodiments, the step of incubating the reaction components is carried out at 30° C.

Thus, in certain embodiments the present invention provides a method for manufacturing a RNA by in vitro transcription (IVT), said method comprising the steps of combining components comprising: (a) a nucleic acid template; (b) a RNA polymerase; and (c) a suitable reaction buffer, the buffer comprising (i) Mg²⁺ at a concentration of 16-50 mM, such as 20-44 mM, 24-40 mM, 28-36 mM, 30-34 mM; (ii) NTPs at a concentration of 20-52 mM, such as 24-46 mM, 30-42, 34-38 mM; and manufacturing the RNA.

In certain embodiments, a method for manufacturing a RNA by in vitro transcription (IVT) comprises the steps of (i) mixing together reaction components comprising: (a) a nucleic acid template; (b) a RNA polymerase; and (c) a suitable reaction buffer, the buffer comprising (i) Mg²⁺ at a concentration of 16-50 mM, such as 20-44 mM, 24-40 mM, 28-36 mM, 30-34 mM; (ii) NTPs at a concentration of 20-52 mM, such as 24-46 mM, 30-42, 34-38 mM; and (ii) incubating the reaction components to produce the RNA.

In certain embodiments, the Mg²⁺ concentration is 28-36 mM, such as 30-34 mM. In certain embodiments the Mg²⁺ concentration is 32 mM. In certain embodiments the Mg²⁺ concentration is 36 mM.

In certain embodiments, the total NTP concentration is 30-42 mM, such as 34-38 mM. In certain embodiments the total NTP concentration is 36 mM.

In certain embodiments, the Mg²⁺ concentration is 16-50 mM, such as 20-44 mM, 24-40 mM, 28-36 mM, 30-34 mM and the total NTP concentration is 30-42 mM, such as 34-38 mM. In certain embodiments, the Mg²⁺ concentration is 28-36 mM, such as 30-34 mM and the total NTP concentration is 30-42 mM, such as 34-38 mM.

In certain embodiments, the Mg²⁺ concentration is 28-36 mM and the total NTP concentration is 30-42 mM. In certain embodiments, the Mg²⁺ concentration is 28-36 mM and the total NTP concentration is 34-38 mM. In certain embodiments, the Mg²⁺ concentration is 30-34 mM and the total NTP concentration is 30-42 mM. In certain embodiments, the Mg²⁺ concentration is 30-34 mM and the total NTP concentration is 34-38 mM.

In certain embodiments, the Mg²⁺ concentration is 32 mM and the total NTP concentration is 36 mM. In certain embodiments the Mg²⁺ concentration is 36 mM and the total NTP concentration is 36 mM.

In certain embodiments, each species of NTP is at the same concentration. The species of NTP may include each of the four RNA precursor compounds ATP, CTP, GTP, and UTP. In certain embodiments, modified NTPs may be included. In certain embodiments not every species of NTP is at the same concentration, for example the concentrations may be adjusted according to the frequency of nucleotide species in the nucleic acid template. In certain embodiments, each species of NTP is at a concentration of 5-13 mM, such as 6-12 mM, 7-11 mM, 8-10 mM. In certain embodiments, each species of NTP is at a concentration of 7-11 mM, such as 8-10 mM. In certain embodiments each species of NTP is at a concentration of 9 mM.

In certain embodiments, for a portion of the duration of IVT reaction, the temperature is 20-40° C., such as 25-35° C., 27-33° C., 28-32° C., 29-31° C. In certain embodiments, for a portion of the duration of the step of manufacturing, the temperature is 30° C. In certain embodiments, the portion is the entire step of manufacturing.

In certain embodiments, a method for manufacturing a RNA by in vitro transcription (IVT) comprises the steps of (i) mixing together reaction components comprising: (a) a nucleic acid template; (b) a RNA polymerase; and (c) a suitable reaction buffer, the buffer comprising (i) Mg²⁺ at a concentration of 16-50 mM, such as 20-44 mM, 24-40 mM, 28-36 mM, 30-34 mM; (ii) NTPs at a concentration of 30-42, such as 34-38 mM; and (ii) incubating the reaction components to produce the RNA.

In certain embodiments, the step of incubating the reaction components is carried out at 20-40° C., such as 25-35° C., 27-33° C., 28-32° C., 29-31° C. in certain embodiments, the step of incubating the reaction components is carried out at 30° C.

In some embodiments, the method for manufacturing a RNA by in vitro transcription (IVT), comprises the steps of combining components comprising: (a) a nucleic acid template; (b) a RNA polymerase; and (c) a suitable reaction buffer, the buffer comprising (i) Mg²⁺ at a concentration of 16-50 mM, such as 20-44 mM, 24-40 mM, 28-36 mM, 30-34 mM; (ii) total NTPs at a concentration of 31-52 mM, such as 32-46 mM, 33-42 mM, 34-38 mM; and manufacturing the RNA.

In certain embodiments the RNA is at least 2,000 nucleotides per strand. In certain embodiments the RNA is at least 5,000 nucleotides per strand, such as 10,000 nucleotides per strand, 15,000 nucleotides per strand, such as at least 20,000 nucleotides per strand.

In some embodiments, the components further comprise an inorganic pyrophosphatase.

Thus, in certain embodiments the method for manufacturing a RNA by in vitro transcription (IVT) comprises (i) a step of combining reaction components comprising a nucleic acid template; a RNA polymerase; an inorganic pyrophosphatase and a reaction buffer, wherein the reaction buffer comprises Mg²⁺ at a concentration of 16-50 mM, such as 20-44 mM, 24-40 mM, 28-36 mM, 30-34 mM and NTPs at a total concentration of 20-52 mM, such as 24-46 mM, 30-42 mM, 34-38 mM, and (ii) a step of incubating the reaction components to produce the RNA.

More particularly, a method for manufacturing a RNA having at least 5,000 nucleotides per strand by in vitro transcription (IVT) comprises (i) a step of combining reaction components comprising a nucleic acid template; a RNA polymerase; an inorganic pyrophosphatase and a reaction buffer, wherein the reaction buffer comprises Mg²⁺ at a concentration of 16-50 mM, such as 20-44 mM, 24-40 mM, 28-36 mM, 30-34 mM and NTPs at a total concentration of 20-52 mM, such as 24-46 mM, 30-42 mM, 34-38 mM, and (ii) a step of incubating the reaction components to produce the RNA.

Certain embodiments comprise, a method for manufacturing a stable and translatable mRNA by in vitro transcription (IVT), said method comprising the steps of (A) combining in a reaction vessel components comprising: (a) a nucleic acid template; (b) a RNA polymerase; and (c) a suitable reaction buffer, the buffer comprising (i) Mg²⁺ at a total concentration of 16-50 mM, such as 20-44 mM, 24-40 mM, 28-36 mM, 30-34 mM; (ii) NTPs at a concentration of 20-52 mM, such as 24-46 mM, 30-42 mM, 34-38 mM; (B) manufacturing the RNA; and (C) capping the 5′ end of the RNA.

Certain embodiments comprise a method for manufacturing a stable and translatable mRNA by in vitro transcription (IVT), said method comprising the steps of (A)

combining in a reaction vessel components comprising: (a) a nucleic acid template; (b) a RNA polymerase; and (c) a suitable reaction buffer, the buffer comprising (i) Mg²⁺ at a total concentration of 16-50 mM, such as 20-44 mM, 24-40 mM, 28-36 mM, 30-34 mM; (ii) NTPs at a concentration of 20-52 mM, such as 24-46 mM, 30-42 mM, 34-38 mM; (B) incubating the components together to produce the RNA; and (C) capping the 5′ end of the RNA.

TriLink CLEANCAP (C₃₂H₄₃N₁₅O₂₄P₄ (free acid)) may be added during the start of the IVT. The capping can be added as part of the manufacturing method. The conditions for the IVT can be changed for the CLEANCAP step.

In certain embodiments, the step (C) of capping the 5′ end of the RNA is carried out at the same time as step (B) incubating the components together to produce the RNA.

In certain embodiments, step (C) capping the 5′ end of the RNA is carried out at after step (B) incubating the components together to produce the RNA.

In certain embodiments, step (C) capping the 5′ end of the RNA comprises incubation of the RNA with an enzymatic capping system, GTP and S-adenosyl methionine.

In certain embodiments, after completion of IVT reaction, 7-methylguanylate cap structure (Cap 0) is added to the 5′end of mRNA using an enzymatic capping system based on the Vaccinia virus Capping Enzyme (VCE). The IVT reaction is added to the capping mix comprising a buffer system with magnesium ions, potassium ions, DTT, GTP, S-adenosyl methionine, and a Vaccinia capping system. The reaction is carried out at 30° C. for 2h.

In certain embodiments, after completion of IVT reaction the mRNA is capped by the technology clean-cap (e.g. TRILINK CLEAN CAP). Cap1 is added to the mRNA.

In a further aspect, the present invention provides a composition for RNA manufacturing comprising: (a) Mg²⁺ at a concentration of 16-50 mM, such as 20-44 mM, 24-40 mM, 28-36 mM, 30-34 mM; (b) NTPs at a total concentration of 20-52 mM, such as 24-46 mM, 30-42 mM, 34-38 mM.

More particularly, the present invention provides a buffer composition for use in a method for manufacturing RNA comprising: (a) Mg²⁺ at a concentration of 16-50 mM, such as 20-44 mM, 24-40 mM, 28-36 mM, 30-34 mM; (b) NTPs at a total concentration of 30-42 mM, such as 34-38 mM.

In certain embodiments, the Mg²⁺ concentration is 28-36 mM, such as 30-34 mM and the total NTP concentration is 30-42 mM, such as 34-38 mM.

In certain embodiments, the Mg²⁺ concentration is 28-36 mM and the total NTP concentration is 30-42 mM. In certain embodiments, the Mg²⁺ concentration is 28-36 mM and the total NTP concentration is 34-38 mM. In certain embodiments, the Mg²⁺ concentration is 30-34 mM and the total NTP concentration is 30-42 mM. In certain embodiments, the Mg²⁺ concentration is 30-34 mM and the total NTP concentration is 34-38 mM.

In certain embodiments, the Mg²⁺ concentration is 32 mM and the total NTP concentration is 36 mM. In certain embodiments the Mg²⁺ concentration is 36 mM and the total NTP concentration is 36 mM.

In certain embodiments the ratio of the concentration of Mg²⁺ and the total NTP concentration is from 0.9:1 to 1.3:1, such as 1:1.

The present invention further provides a method for manufacturing a RNA by in vitro transcription (IVT), said method comprising the steps of (A) combining in a reaction vessel components comprising: (a) a nucleic acid template; (b) a RNA polymerase; and (c) a suitable reaction buffer, the buffer comprising (i) Mg²⁺ at a concentration of 32 mM; (ii) NTPs at a total concentration of 36 mM; (B) manufacturing the RNA; and (C) capping the 5′ end of the RNA.

In certain embodiments, the Mg²⁺ concentration is 16-50 mM, such as 20-44 mM, 24-40 mM, 28-36 mM, 30-34 mM and the total NTP concentration is 30-42 mM, such as 34-38 mM. In certain embodiments, the Mg²⁺ concentration is 28-36 mM, such as 30-34 mM and the total NTP concentration is 30-42 mM, such as 34-38 mM.

In certain embodiments, the Mg²⁺ concentration is 28-36 mM and the total NTP concentration is 30-42 mM. In certain embodiments, the Mg²⁺ concentration is 28-36 mM and the total NTP concentration is 34-38 mM. In certain embodiments, the Mg²⁺ concentration is 30-34 mM and the total NTP concentration is 30-42 mM. In certain embodiments, the Mg²⁺ concentration is 30-34 mM and the total NTP concentration is 34-38 mM.

In certain embodiments, the Mg²⁺ concentration is 32 mM and the total NTP concentration is 36 mM. In certain embodiments the Mg²⁺ concentration is 36 mM and the total NTP concentration is 36 mM.

In a further aspect, the present invention provides a method for manufacturing a RNA by in vitro transcription (IVT) comprising the steps of (i) mixing together reaction components comprising: (a) a nucleic acid template; (b) a RNA polymerase; and (c) a suitable reaction buffer, the buffer comprising (i) Mg²⁺ at a concentration of 16-50 mM, such as 20-44 mM, 24-40 mM, 28-36 mM, 30-34 mM; (ii) NTPs at a total concentration of 20-52 mM, such as 24-46 mM, 30-42, 34-38 mM; wherein the ratio of the concentration of Mg²⁺ and the concentration of NTPs is from 0.9:1 to 1.3:1, and (ii) incubating the reaction components to produce the RNA.

In some embodiments of the invention, a method is provided for manufacturing a RNA by IVT comprising (i) a step of combining reaction components comprising a nucleic acid template; a RNA polymerase; and a reaction buffer, wherein the reaction buffer comprises Mg²⁺ at a concentration of 28-36 mM, such as 30-34 mM, and NTPs at a total concentration of 30-42 mM, such as 34-38 mM, and (ii) a step of incubating the reaction components at 30° C. to produce the RNA, wherein the ratio of the concentration of Mg²⁺ and the total NTP concentration is 1:1.

In certain embodiments the Mg²⁺ concentration is 36 mM and the total NTP concentration is 36 mM.

More particularly, a method for manufacturing a RNA by in vitro transcription (IVT), wherein the RNA has at least 5,000 nucleotides per strand, such as 10,000 nucleotides per strand, 15,000 nucleotides per strand, 20,000 nucleotides per strand, 25,000 nucleotides per strand, comprises (i) a step of combining reaction components comprising a nucleic acid template; a RNA polymerase; an inorganic pyrophosphatase and a reaction buffer, wherein the reaction buffer comprises Mg²⁺ at a concentration of 16-50 mM, such as 20-44 mM, 24-40 mM, 28-36 mM, 30-34 mM and NTPs at a total concentration of 20-52 mM, such as 24-46 mM, 30-42 mM, 34-38 mM, and (ii) a step of incubating the reaction components to produce the RNA.

In certain embodiments, the Mg²⁺ concentration is 16-50 mM, such as 20-44 mM, 24-40 mM, 28-36 mM, 30-34 mM and the total NTP concentration is 30-42 mM, such as 34-38 mM. In certain embodiments, the Mg²⁺ concentration is 28-36 mM, such as 30-34 mM and the total NTP concentration is 30-42 mM, such as 34-38 mM.

In certain embodiments, the Mg²⁺ concentration is 28-36 mM and the total NTP concentration is 30-42 mM. In certain embodiments, the Mg²⁺ concentration is 28-36 mM and the total NTP concentration is 34-38 mM. In certain embodiments, the Mg²⁺ concentration is 30-34 mM and the total NTP concentration is 30-42 mM. In certain embodiments, the Mg²⁺ concentration is 30-34 mM and the total NTP concentration is 34-38 mM.

In certain embodiments, the Mg²⁺ concentration is 32 mM and the total NTP concentration is 36 mM. In certain embodiments the Mg²⁺ concentration is 36 mM and the total NTP concentration is 36 mM. In certain embodiments, the step of incubating the reaction components is carried out at 20-40° C., such as 25-35° C., 27-33° C., 28-32° C., 29-31° C. In certain embodiments, the step of incubating the reaction components is carried out at 30° C. In certain embodiments the ratio of the concentration of Mg²⁺ and the total NTP concentration is from 0.9:1 to 1.3:1, such as 1:1.

In some embodiments of the invention, a method is provided for manufacturing a RNA by IVT, wherein the RNA has at least 15,000 nucleotides per strand, comprising (i) a step of combining reaction components comprising a nucleic acid template; a RNA polymerase; an inorganic pyrophosphatase and a reaction buffer, wherein the reaction buffer comprises Mg²⁺ at a concentration of 28-36 mM and NTPs at a total concentration of 34-38 mM, wherein the ratio of the concentration of Mg²⁺ and the total NTP concentration is 1:1, and (ii) a step of incubating the reaction components at 30° C. to produce the RNA.

The conditions for manufacturing mRNA via IVT may change depending on the size of the mRNA (self-replicating vs non-replicating). Therefore, concentrations, temperature, etc. may be different depending on the type of mRNA.

Non-Replicating mRNA

In some embodiments of the invention, a method is provided for manufacturing a RNA by IVT comprising a step of combining components comprising a nucleic acid template; a RNA polymerase; and a reaction buffer, wherein the reaction buffer comprises Mg²⁺ at a concentration of 5-48 mM, such as 6-30 mM, 7-25 mM, 8-20 mM and NTPs at a total concentration of 8-40 mM, such as 12-28 mM, 12-20 and a step of manufacturing the RNA. In certain embodiments, the Mg²⁺ concentration is 10-20 mM and the total NTPs concentration is 12-24 mM. In certain embodiments, the Mg²⁺ concentration is 16 mM and total NTPs concentration is at 20 mM. In certain embodiments, each species of NTP is at the same concentration. In certain embodiments, each species of NTP is at a concentration of 2-8 mM. In certain embodiments, each species of NTP is at a concentration of 5 mM. In certain embodiments the ratio of the concentration of Mg²⁺ and the total species NTP concentration is from 2:1 to 24:1, such as 3.2:1. In certain embodiments, the Mg²⁺:NTP species ratio is 3.2:1. In certain embodiments, for a portion of the duration of manufacturing, the temperature is 20-50° C., such as 25-45° C., 30-40° C., 35-40° C. In certain embodiments, for a portion of the duration of the step of manufacturing, the temperature is 37° C. In certain embodiments, the mRNA is non-replicating. In some embodiments, the method further comprising a step of capping the 5′ end of the RNA. In certain embodiments, the step of capping is performed during the IVT process. In certain embodiments, the step of capping is co-transcriptional capping. In certain embodiments, the components further comprises CLEANCAP (C₃₂H₄₃N₁₅O₂₄P₄ (free acid)) AG. In certain embodiments, the CLEANCAP AG concentration is 0-20 mM, such as 0-15 mM, 0-10 mM, 2-10 mM, 2-8 mM. In certain embodiments, the CLEANCAP AG concentration is 8 mM. In certain embodiments, the suitable reaction buffer further comprising pyrophosphatase, Rnase inhibitor, DTT, and spermidine.

In some embodiments of the invention, a composition is provided comprising: (a) Mg²⁺ at a concentration of 5-48 mM, such as 6-30 mM, 7-25 mM, 8-20 mM; (b) NTPs at a total concentration of 8-40 mM, such as 12-28 mM. In certain embodiments, the Mg²⁺ concentration is 10-20 mM and the total NTPs concentration is 12-24 mM. In certain embodiments, the Mg²⁺ concentration is 16 mM and total NTPs concentration is at 20 mM. In certain embodiments, each species of NTP is at the same concentration. In certain embodiments, each species of NTP is at a concentration of 2-8 mM. In certain embodiments, each species of NTP is at a concentration of 5 mM. In certain embodiments the ratio of the concentration of Mg²⁺ and the total species NTP concentration is from 2:1 to 24:1, such as 3.2:1. In certain embodiments, the Mg²⁺:NTP species ratio is 3.2:1. In certain embodiments, for a portion of the duration of manufacturing, the temperature is 20-50° C., such as 25-45° C., 30-40° C., 35-40° C. In certain embodiments, for a portion of the duration of the step of manufacturing, the temperature is 37° C. In certain embodiments, the mRNA is non-replicating. In some embodiments, the method further comprising a step of capping the 5′ end of the RNA. In certain embodiments, the step of capping is performed during the IVT process. In certain embodiments, the step of capping is co-transcriptional capping. In certain embodiments, the components further comprises CLEANCAP (C₃₂H₄₃N₁₅O₂₄P₄ (free acid)) AG. In certain embodiments, the CLEANCAP AG concentration is 0-20 mM, such as 0-15 mM, 0-10 mM, 2-10 mM, 2-8 mM. In certain embodiments, the CLEANCAP AG concentration is 8 mM. In certain embodiments, the suitable reaction buffer further comprising pyrophosphatase, Rnase inhibitor, DTT, and spermidine.

In certain embodiments of the invention, a method is provided for manufacturing a stable and translatable mRNA by in vitro transcription (IVT), said method comprising the steps of (A) combining in a reaction vessel components comprising: (a) a nucleic acid template; (b) a RNA polymerase; and (c) a suitable reaction buffer, the buffer comprising (i) Mg²⁺ at a total concentration of 5-48 mM, such as 6-30 mM, 7-25 mM, 8-20 mM; (ii) NTPs at a concentration of 8-40 mM, such as 12-28 mM; (B) manufacturing the RNA. In certain embodiments, the Mg²⁺ concentration is 16 mM and total NTPs concentration is at 20 mM. In certain embodiments, each species of NTP is at the same concentration. In certain embodiments, each species of NTP is at a concentration of 2-8 mM. In certain embodiments, each species of NTP is at a concentration of 5 mM. In certain embodiments the ratio of the concentration of Mg²⁺ and the total species NTP concentration is from 2:1 to 24:1, such as 3.2:1. In certain embodiments, the Mg²⁺:NTP species ratio is 3.2:1. In certain embodiments, for a portion of the duration of manufacturing, the temperature is 20-50° C., such as 25-45° C., 30-40° C., 35-40° C. In certain embodiments, for a portion of the duration of the step of manufacturing, the temperature is 37° C. In certain embodiments, the mRNA is non-replicating. In some embodiments, the method further comprising a step of capping the 5′ end of the RNA. In certain embodiments, the step of capping is performed during the IVT process. In certain embodiments, the step of capping is co-transcriptional capping. In certain embodiments, the components further comprises CLEANCAP (C₃₂H₄₃N₁₅O₂₄P₄ (free acid)) AG. In certain embodiments, the CLEANCAP AG concentration is 0-20 mM, such as 0-15 mM, 0-10 mM, 2-10 mM, 2-8 mM. In certain embodiments, the CLEANCAP AG concentration is 8 mM. In certain embodiments, the suitable reaction buffer further comprising pyrophosphatase, Rnase inhibitor, DTT, and spermidine.

In certain embodiments of the invention, a method is provided for manufacturing a pharmaceutically acceptable RNA by in vitro transcription (IVT), said method comprising the steps of (A) combining in a reaction vessel components comprising: (a) a nucleic acid template; (b) a RNA polymerase; and (c) a suitable reaction buffer, the buffer comprising (i) Mg²⁺ at a total concentration of 5-48 mM, such as 6-30 mM, 7-25 mM, 8-20 mM; (ii) NTPs at a concentration of 8-40 mM, such as 12-28 mM; (B) manufacturing the RNA; (C) purifying and packaging the RNA. In certain embodiments, the Mg²⁺ concentration is 16 mM and total NTPs concentration is at 20 mM.

In certain embodiments, each species of NTP is at the same concentration. In certain embodiments, each species of NTP is at a concentration of 2-8 mM. In certain embodiments, each species of NTP is at a concentration of 5 mM. In certain embodiments the ratio of the concentration of Mg²⁺ and the total species NTP concentration is from 2:1 to 24:1, such as 3.2:1. In certain embodiments, the Mg²⁺:NTP species ratio is 3.2:1. In certain embodiments, for a portion of the duration of manufacturing, the temperature is 20-50° C., such as 25-45° C., 30-40° C., 35-40° C. In certain embodiments, for a portion of the duration of the step of manufacturing, the temperature is 37° C. In certain embodiments, the mRNA is non-replicating. In some embodiments, the method further comprising a step of capping the 5′ end of the RNA. In certain embodiments, the step of capping is performed during the IVT process. In certain embodiments, the step of capping is co-transcriptional capping. In certain embodiments, the components further comprises CLEANCAP (C₃₂H₄₃N₁₅O₂₄P₄ (free acid)) AG. In certain embodiments, the CLEANCAP AG concentration is 0-20 mM, such as 0-15 mM, 0-10 mM, 2-10 mM, 2-8 mM. In certain embodiments, the CLEANCAP AG concentration is 8 mM. In certain embodiments, the suitable reaction buffer further comprising pyrophosphatase, Rnase inhibitor, DTT, and spermidine.

In certain embodiments, a method is provided for manufacturing a pharmaceutically acceptable mRNA by in vitro transcription (IVT), said method comprising the steps of (A) combining in a reaction vessel components comprising: (a) a nucleic acid template; (b) a RNA polymerase; and (c) a suitable reaction buffer, the buffer comprising (i) Mg²⁺ at a total concentration of 5-48 mM, such as 6-30 mM, 7-25 mM, 8-20 mM; (ii) NTPs at a concentration of 8-40 mM, such as 12-28 mM; (B) manufacturing the RNA; (C) capping the 5′ end of the RNA; (D) purifying and packaging the capped RNA. In certain embodiments, the Mg²⁺ concentration is 16 mM and total NTPs concentration is at 20 mM. In certain embodiments, each species of NTP is at the same concentration. In certain embodiments, each species of NTP is at a concentration of 2-8 mM. In certain embodiments, each species of NTP is at a concentration of 5 mM. In certain embodiments the ratio of the concentration of Mg²⁺ and the total species NTP concentration is from 2:1 to 24:1, such as 3.2:1. In certain embodiments, the Mg²⁺:NTP species ratio is 3.2:1. In certain embodiments, for a portion of the duration of manufacturing, the temperature is 20-50° C., such as 25-45° C., 30-40° C., 35-40° C. In certain embodiments, for a portion of the duration of the step of manufacturing, the temperature is 37° C. In certain embodiments, the mRNA is non-replicating. In some embodiments, the method further comprising a step of capping the 5′ end of the RNA. In certain embodiments, the step of capping is performed during the IVT process. In certain embodiments, the step of capping is co-transcriptional capping. In certain embodiments, the components further comprises CLEANCAP (C₃₂H₄₃N₁₅O₂₄P₄ (free acid)) AG. In certain embodiments, the CLEANCAP AG concentration is 0-20 mM, such as 0-15 mM, 0-10 mM, 2-10 mM, 2-8 mM. In certain embodiments, the CLEANCAP AG concentration is 8 mM. In certain embodiments, the suitable reaction buffer further comprising pyrophosphatase, Rnase inhibitor, DTT, and spermidine.

In certain embodiments, each species of NTP is at the same concentration. The species of NTP may include each of the four RNA precursor compounds ATP, CTP, GTP, and UTP. In certain embodiments, modified NTPs may be included. In certain embodiments not every species of NTP is at the same concentration, for example the concentrations may be adjusted according to the frequency of nucleotide species in the nucleic acid template. In certain embodiments, each species of NTP is at a concentration of 2-8 mM. In certain embodiments each species of NTP is at a concentration of 5 mM.

In certain embodiments, for a portion of the duration of IVT reaction, the temperature is 20-50° C., such as 25-45° C., 30-40° C., 35-40° C. In certain embodiments, for a portion of the duration of the step of manufacturing, the temperature is 37° C. In certain embodiments, the portion is the entire step of manufacturing.

In certain embodiments the RNA is at least 2,000 nucleotides per strand. In certain embodiments the RNA is at least 5,000 nucleotides per strand, such as 10,000 nucleotides per strand, 15,000 nucleotides per strand, such as at least 20,000 nucleotides per strand.

In some embodiments, the components further comprise an inorganic pyrophosphatase.

Thus, in certain embodiments the method for manufacturing a RNA by in vitro transcription (IVT) comprises (i) a step of combining reaction components comprising a nucleic acid template; a RNA polymerase; an inorganic pyrophosphatase and a reaction buffer, wherein the reaction buffer comprises Mg²⁺ at a concentration of 5-48 mM, such as 6-30 mM, 7-25 mM, 8-20 mM, and (ii) a step of incubating the reaction components to produce the RNA.

More particularly, a method for manufacturing a RNA having at least 5,000 nucleotides per strand by in vitro transcription (IVT) comprises (i) a step of combining reaction components comprising a nucleic acid template; a RNA polymerase; an inorganic pyrophosphatase and a reaction buffer, wherein the reaction buffer comprises Mg²⁺ at a concentration of 5-48 mM, such as 6-30 mM, 7-25 mM, 8-20 mM and NTPs at a total concentration of 8-40 mM, such as 12-28 mM, and (ii) a step of incubating the reaction components to produce the RNA.

Certain embodiments comprise, a method for manufacturing a stable and translatable mRNA by in vitro transcription (IVT), said method comprising the steps of (A) combining in a reaction vessel components comprising: (a) a nucleic acid template; (b) a RNA polymerase; and (c) a suitable reaction buffer, the buffer comprising (i) Mg²⁺ at a total concentration of 5-48 mM, such as 6-30 mM, 7-25 mM, 8-20 mM; (ii) NTPs at a concentration of 8-40 mM, such as 12-28 mM; (B) manufacturing the RNA; and (C) capping the 5′ end of the RNA.

Certain embodiments comprise a method for manufacturing a stable and translatable mRNA by in vitro transcription (IVT), said method comprising the steps of (A)

combining in a reaction vessel components comprising: (a) a nucleic acid template; (b) a RNA polymerase; and (c) a suitable reaction buffer, the buffer comprising (i) Mg²⁺ at a total concentration of 5-48 mM, such as 6-30 mM, 7-25 mM, 8-20 mM; (ii) NTPs at a concentration of 8-40 mM, such as 12-28 mM; (B) incubating the components together to produce the RNA; and (C) capping the 5′ end of the RNA.

TriLink CLEANCAP (C₃₂H₄₃N₁₅O₂₄P₄ (free acid)) may be added during the start of the IVT. The capping can be added as part of the manufacturing method. The conditions for the IVT can be changed for the CLEANCAP step.

In certain embodiments, the step (C) of capping the 5′ end of the RNA is carried out at the same time as step (B) incubating the components together to produce the RNA.

In certain embodiments, step (C) capping the 5′ end of the RNA is carried out at after step (B) incubating the components together to produce the RNA.

In certain embodiments, step (C) capping the 5′ end of the RNA comprises incubation of the RNA with an enzymatic capping system, GTP and S-adenosyl methionine.

In certain embodiments, after completion of during the IVT reaction, 7-methylguanylate cap structure (Cap 1) is added to the 5′ end of mRNA using an enzymatic capping system based on the Vaccinia virus Capping Enzyme (VCE). The IVT reaction is added to the capping mix comprising a buffer system with magnesium ions, potassium ions, DTT, GTP, S-adenosyl methionine, and a Vaccinia capping system. The reaction is carried out at 37° C.

In certain embodiments, during IVT reaction the mRNA is capped by the technology clean-cap (e.g. TRILINK CLEAN CAP). Cap1 is added to the mRNA.

In a further aspect, the present invention provides a method for manufacturing a RNA by in vitro transcription (IVT) comprising the steps of (i) mixing together reaction components comprising: (a) a nucleic acid template; (b) a RNA polymerase; and (c) a suitable reaction buffer, the buffer comprising (i) Mg²⁺ at a concentration of 5-48 mM, such as 6-30 mM, 7-25 mM, 8-20 mM; (ii) NTPs at a total concentration of 8-40 mM, such as 12-28 mM; wherein the ratio of the concentration of Mg²⁺ and the concentration of species of NTPs is from, and (ii) incubating the reaction components to produce the RNA.

In some embodiments of the invention, a method is provided for manufacturing a RNA by IVT comprising (i) a step of combining reaction components comprising a nucleic acid template; a RNA polymerase; and a reaction buffer, wherein the reaction buffer comprises Mg²⁺ at a concentration of 5-48 mM, such as 6-30 mM, 7-25 mM, 8-20 mM, and NTPs at a total concentration of 8-40 mM, such as 12-28 mM, and (ii) a step of incubating the reaction components at 37° C. to produce the RNA, wherein the ratio of the concentration of Mg²⁺ and the total NTP concentration is 1:3.2.

More particularly, a method for manufacturing a RNA by in vitro transcription (IVT), wherein the RNA has at least 500 nucleotides per strand, such as 1,000-6,000 nucleotides per strand, 2,000-5, 500 nucleotides per strand, 3,000-5,000 nucleotides per strand, 4,000-5,000 nucleotides per stand, 4.500-5,000 nucleotides per strand comprises (i) a step of combining reaction components comprising a nucleic acid template; a RNA polymerase; an inorganic pyrophosphatase and a reaction buffer, wherein the reaction buffer comprises Mg²⁺ at a concentration of 5-48 mM, such as 5-30 mM, 5-25 mM, 5-20 mM and NTPs at a total concentration of 8-40 mM, such as 12-28 mM, and (ii) a step of incubating the reaction components to produce the RNA.

In certain embodiments, the Mg²⁺ concentration is 16 mM and the total NTP concentration is 20 mM. In certain embodiments, the step of incubating the reaction components is carried out at 20-50° C., such as 25-45° C., 30-40° C., 35-40° C. In certain embodiments, the step of incubating the reaction components is carried out at 37° C. In certain embodiments the ratio of the concentration of Mg²⁺ and the species NTP concentration is from 2:1 to 24:1, such as 1:3.2.

In some embodiments of the invention, a method is provided for manufacturing a RNA by IVT, wherein the RNA has at least 1,000 nucleotides per strand, comprising (i) a step of combining reaction components comprising a nucleic acid template; a RNA polymerase; an inorganic pyrophosphatase and a reaction buffer, wherein the reaction buffer comprises Mg²⁺ at a concentration of 10-20 mM and NTPs at a total concentration of 12-24 mM, wherein the ratio of the concentration of Mg²⁺ and the total NTP concentration is 1.25:1, and (ii) a step of incubating the reaction components at 37° C. to produce the RNA.

The invention will be further described by reference to the following, non-limiting, figures and examples.

### EXAMPLES

It is understood that the examples and embodiments described herein are for illustrative purposes only and that various modifications or changes in light thereof will be suggested to persons skilled in the art and are to be included within the spirit and purview of this application.

RNA may be produced by in vitro transcription (IVT) of linearized pDNA template using a T7 RNA polymerase. RNA may be purified to remove enzymes, pDNA template and small molecules using chromatography and tangential flow filtration (TFF). A one factor at a time (OFAT) approach may be used during the development and improvement of the IVT process. The OFAT approach considers the effects of individual enzymatic reaction components on the yield and quality of RNA.

A design of experiment (DoE) approach was taken to select the conditions for the IVT reaction for long mRNAs (up to 15kb) and shorter mRNAs (1000 nt to 5000 nt). DoE study can determine the relationship between various factors that influences a process or an output of the process. Here, we used definitive screening designs and central composite designs to identify the factors that have significant influence on maximizing the yield and quality of mRNA (percentage of full length of mRNA in total RNA synthesized) as well as established a mathematical relationship between each factor impacting the yield and quality of mRNA. First, a definitive screening was conducted to determine the components that have the highest significance in IVT and to identify their secondary interactions. Next, central composite design was used to adjust and analyze curvature of the design space.

### Example 1: Plasmid DNA Synthesis and Purification

The plasmid DNA (pDNA) used for this study was produced using Escherichia coli culture. The pDNA was extracted and purified using a Plasmid giga prep kit from Qiagen. pDNA was then linearized using restriction enzyme, purified with ethanol precipitation and then used as template in IVT reaction.

### Example 2: Design of Experiment (DoE)

The experiments were designed and analyzed in JMP software using definitive screening design and central composite design with RNA yield and percent of full-length mRNA in total RNA as the responses. The standard least squares fit model was used to analyze the data of each DoE.

### Example 3: Self-Replicating RNA Synthesis and Quantification

All IVT reactions were carried out using components listed in table 1 with a final volume of 100 μL per reaction incubated for 5 h. For definitive screening, all components except RNase Inhibitor were varied at low, medium and high concentration. For central composite design, five levels of each of the factors—magnesium chloride (MgCl₂), Nucleotide triphosphate (NTP), Tris-HCl and Temperature were used. RNAs were precipitated using lithium chloride after completion of IVT and quantified using Nanodrop at 260 nm wavelength. The percentage of full-length mRNA in total RNA transcribed (quality of RNA) was quantified using LabChip GXII Touch (Perkin Elmer) using manufacturer's protocol.

TABLE 1
List of IVT reaction components

| | | Storage |
|---|---|---|
| Item | Vendor | Temperature |
| Nuclease free water | N/A | Ambient |
| Tris-HCl pH 7.0 (1M) | Sigma-Aldrich | Ambient |
| Magnesium chloride (2M) | Sigma-Aldrich | Ambient |
| Adenosine-5′-triphosphate (100 mM) | NEB | −20° C. |
| Cytosine-5′-triphosphate (100 mM) | NEB | −20° C. |
| Guanosine-5′-triphosphate (100 mM) | NEB | −20° C. |
| Uridine-5′-triphosphate (100 mM) | NEB | −20° C. |
| DL-dithiothreitol (DTT) (1M) | Sigma-Aldrich | −20° C. |
| Spermidine (100 mM) | Sigma-Aldrich | +4° C. |
| Pyrophosphatase, inorganic (100 U/mL) | NEB | −20° C. |
| RNase inhibitor, human (40,000 U/mL) | NEB | −20° C. |
| T7 RNA polymerase (50,000 U/mL) | NEB | −20° C. |
| Linear DNA template (1 mg/mL) | N/A | −20° C. |

### Example 4: The Factors with the Highest Significance on IVT of Self-Replicating RNA were Identified Using Definitive Screening Design

A definitive screening design of experiment was designed with nine factors, where three different levels (a low, medium, and high point) per factor were tested (Table 2) for self-amplifying RNA. A minimum number of reactions (22 in total) was performed to screen for effects of individual factors and their interactions, including quadratic effects on RNA yield and quality. RNase Inhibitor concentration was kept fixed in all reactions at 1,000 U/mL. The RNA yield and percent of full-length RNA quantified were entered to the JMP software and a fit model of standard least squares was generated.

TABLE 2
The components used for a definitive screening design DoE

| Factors | Levels used |
|---|---|
| IVT Temperature (° C.) | 24, 30.5, 37 |
| Template DNA (ug/mL) | 20, 35, 50 |
| T7 RNA Polymerase (U/mL) | 2500, 3750, 5000 |
| Magnesium chloride (mM) | 16, 24, 32 |
| Nucleotide (mM) | 1, 5, 9 |
| DTT (mM) | 6, 13, 20 |
| Tris-HCI pH 7 (mM) | 20, 40, 60 |
| Pyrophosphatase (U/mL) | 2, 5, 8 |
| Spermidine (mM) | 0, 2.5, 5 |
| RNase Inhibitor (U/mL) | 1000 |

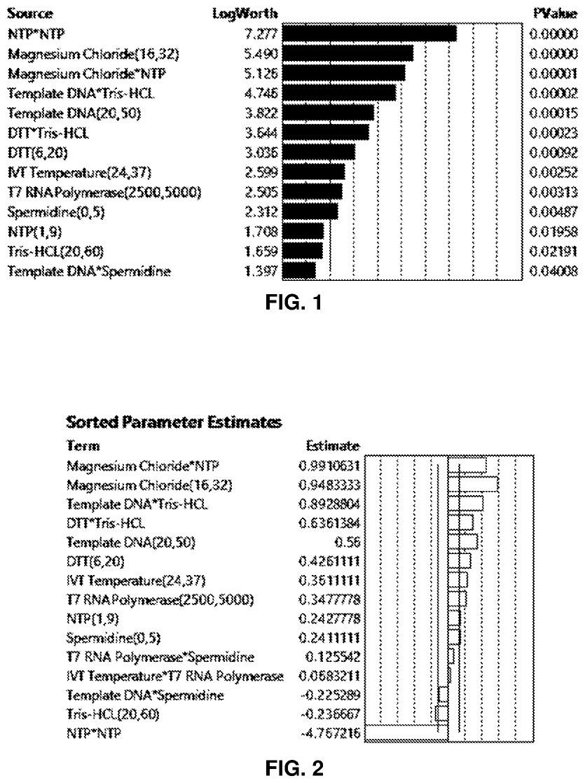

The significance of each factor and their interactions are shown in FIG. 1. The factors and interactions with a logworth >2 or P-value <0.005 is considered significant. Magnesium Ion and magnesium-NTP interaction have the highest significance in the overall IVT reaction.

The effect of each factor and their interactions on both the RNA yield and RNA quality was identified. As indicated by the coefficient estimates, magnesium chloride and magnesium-NTP interaction have the highest impact on the IVT yield (FIG. 2). Other factors such as DNA template, IVT temperature, T7 RNA polymerase also have positive impact on the IVT yield.

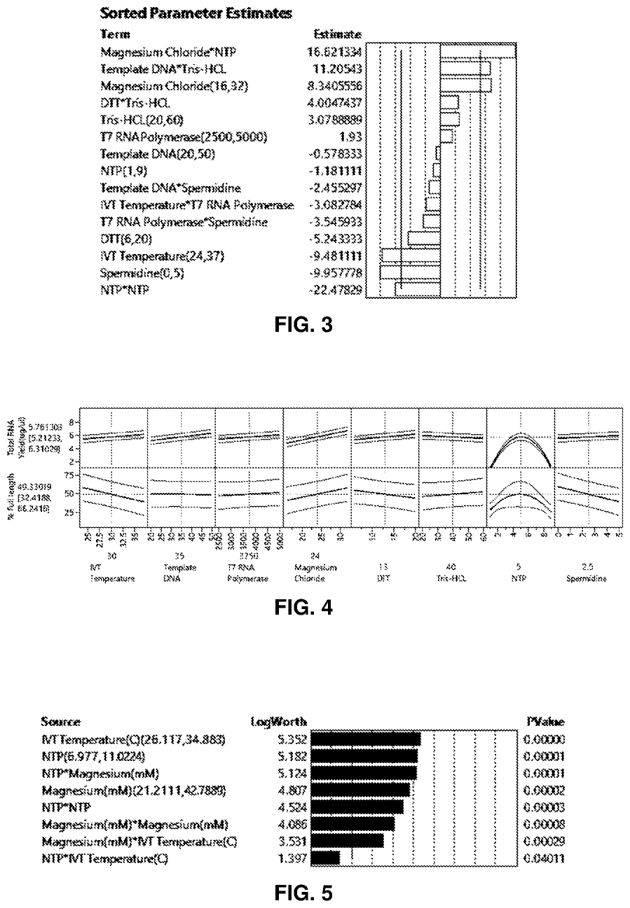

Similar to RNA yield, the interaction between magnesium and NTP also has the highest effect on the RNA quality (FIG. 3). In contrast, IVT temperature, spermidine and NTP-NTP interactions have a significant negative impact on the RNA quality resulting in higher amount of truncated RNA species.

The effect of each of the factors on RNA yield and RNA quality to comprehend the relationship between each factor and their impact on overall IVT performance (FIG. 4) was plotted. With an increase in IVT temperature from 25° C. to 37° C., there was a gradual increase in the IVT yield. However, the RNA quality drops significantly as expected because an increase in temperature leads to non-specific hydrolysis of phosphodiester bonds. A similar trend was observed for spermidine concentration and DTT concentration.

The spermidine and DTT concentrations were fixed in the moderate level at 2 mM and 10 mM respectively to ensure that the RNA quality is not compromised. Tris-HCl concentration does not have significant impact on both the yield and the quality of RNA. In contrast, increasing the concentrations of both DNA template and T7 RNA polymerase led to moderate increase in the RNA yield and RNA quality. The concentrations of both DNA template and T7 RNA polymerase fixed at high level (50 ug/mL and 5000 U/mL respectively) for central composite design study.

Magnesium Ion and NTP concentrations have the most significant impact on both the yield and the quality of RNA. Both the yield and the quality of RNA increased as magnesium concentration increased from 16 mM to 32 mM. In contrast, the RNA yield and quality increased sharply with an increase in NTP concentration up to 5 mM, and then both the yield and quality dropped significantly above 5 mM demonstrating a quadratic effect.

The impact of interactions between different factors on the IVT yield and the RNA quality was studied. Interactions between most of the factors were not observed to impact RNA yield and quality, as demonstrated by an increase in the level of an individual factor having either a positive or negative impact irrespective of the other factor. For example, RNA yield and quality increase with an increase in DNA template concentration irrespective of the IVT temperature used. The interactions of Tri-HCl concentration with DTT and DNA template concentrations did, however, have an impact on the IVT. In the case of both DNA template and DTT, increase in concentration of Tris-HCl at higher concentrations of either DNA template or DTT had positive impact on the IVT, as demonstrated by increase in the yield and quality. In contrast, the RNA yield and quality decreased with increase in Tri-HCl concentration at lower concentrations of DNA template and DTT.

The interaction between magnesium ion and NTP have significant impact on the IVT. We observed that the increase in magnesium ion concentration at lower level of NTP (1 mM) had deleterious effect on the IVT. However, the increase in magnesium ion concentration at higher level of NTP (9 mM) resulted in increase in the RNA yield and quality. The surface response for IVT yield with an increase in NTP concentration at different level of magnesium ion concentration was found to be asymmetrical. For example, the rate at which IVT yield changes at 16 mM Mg²⁺ with change in NTP concentration is different than the rate at which IVT yield changes at 32 mM Mg²⁺ with change in NTP concentration.

### Example 5: Central Composite Design was Conducted with Significant Factors (Magnesium, NTP and IVT Temperature) to Further Adjust IVT Conditions

Further adjustments of IVT was done by performing a central composite design of experiment with four factors (magnesium ion concentration, NTP concentration, IVT temperature and Tris-HCl concentration) where five different levels per factor were tested (Table 3). All other factors are kept fixed across all the reactions. A total of 28 reactions was performed to adjust the IVT for the self-amplifying RNA yield and quality. The RNA yield and percent of full-length RNA quantified were entered to the JMP software and a fit model of standard least squares was generated as previously done for definitive screening design.

TABLE 3
The components used for a central composite design DoE

| Factors | Levels used |
|---|---|
| IVT Temperature (° C.) | 24, 26.1, 30.5, 34.9, 37 |
| Magnesium chloride (mM) | 16, 21.2, 32, 42.8, 48 |
| Nucleotide (mM) | 6, 7, 9, 11, 12 |
| Tris-HCl pH 7 (mM) | 20, 26.5, 40, 53.5, 60 |
| Template DNA (ug/mL) | 50 |
| T7 RNA Polymerase (U/mL) | 5000 |
| DTT (mM) | 10 |
| Pyrophosphatase (U/mL) | 2 |
| Spermidine (mM) | 2 |
| RNase Inhibitor (U/mL) | 1000 |

The significant factors and interactions with a logworth >2 or P-value <0.005 are shown in FIG. 5. Magnesium ion concentration and magnesium-NTP interaction have the highest significance in the overall IVT reaction.

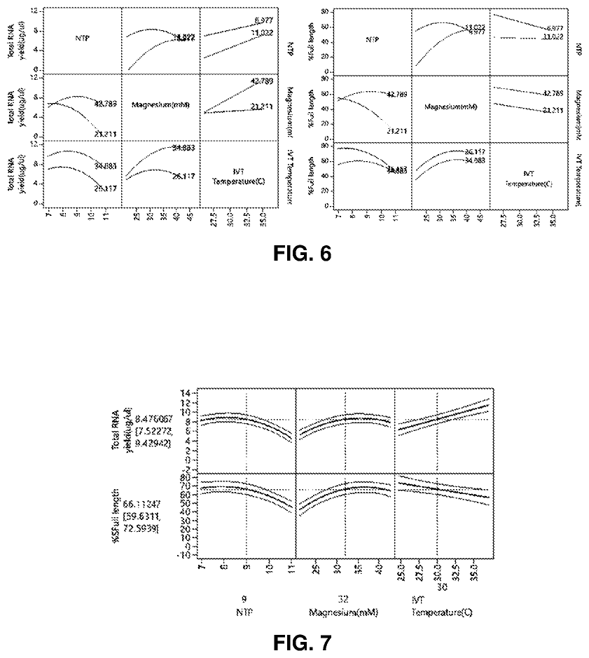

Interaction profiles were plotted between the three factors on IVT yield (FIG. 6) and RNA quality (FIG. 7). The five different levels allowed the interaction profiles of high resolution to be obtained.

Further examination of interaction between magnesium ion and NTP concentrations indicated that the highest RNA yield and quality was achieved with magnesium ion concentration of 36 mM and NTP concentration of 9 mM each with a ratio of 1:1 between total NTP and magnesium (FIG. 7). Further increasing the concentration of each NTP or magnesium had a deleterious effect on IVT, causing either RNA yield or RNA quality to decrease. Increase in IVT temperature increase the RNA yield while decreasing RNA quality. Therefore, moderate IVT temperature of 30° C. is chosen to maintain high yield and low degree of hydrolysis.

DoE approach was utilized to adjust the IVT process for long mRNA so that maximal RNA yield and quality can be achieved. Magnesium ions play an important role in IVT reaction, especially their interaction with NTPs. The right ratio (1:1 between magnesium and total NTPs) of these two components is needed to yield high RNA concentrations in high quality.

### Example 6: The Factors with the Highest Significance on IVT were Identified Using Definitive Screening Design

A definitive screening design of experiment was designed with factors, where different levels (a low, medium, and high point) per factor were tested (Table 4) for non-replicating RNA. A minimum number of reactions was performed to screen for effects of individual factors and their interactions, including quadratic effects on RNA yield and quality. RNase Inhibitor concentration was kept fixed in all reactions at 1,000 U/mL. The RNA yield and percent of full-length RNA quantified were entered to the JMP software and a fit model of standard least squares was generated.

TABLE 4
The components used for a definitive screening design DoE

| Factors | Levels used |
|---|---|
| IVT Temperature (° C.) | 30, 33.5, 37 |
| Template DNA (ug/mL) | 20, 35, 50 |
| T7 RNA Polymerase (μL) | 0, 5, 10 |
| Magnesium chloride (μL) | 0, 4, 8, 16, 32, 48 |
| Nucleotide (mM) | 1, 5, 9 |
| DTT (mM) | 6, 13, 20 |
| Tris-HCl pH 7 (mM) | 20, 40, 60 |
| Pyrophosphatase (U/mL) | 2, 5, 8 |
| Spermidine (mM) | 0, 2.5, 5 |
| RNase Inhibitor (U/mL) | 1000 |

### Example 7: Non-Replicating RNA Synthesis and Quantification

All IVT reactions were carried out using components listed in Table 5 with a final volume of 100 μL per reaction incubated for various times. For definitive screening, all components were varied at low, medium and high concentration. For optimal designs, five levels of each of the factors—magnesium chloride (MgCl₂), Nucleotide triphosphate (NTP), and CLEANCAP AG were used for one study, and three levels of time vs. temperature were used for another. RNAs were precipitated using lithium chloride after completion of IVT and quantified using Nanodrop at 260 nm wavelength. The percentage of full-length mRNA in total RNA transcribed (quality of RNA) was quantified using LabChip GXII Touch (Perkin Elmer) using manufacturer's protocol. The percentage of dsRNA was determined using an in-house Luminex binding assay.

TABLE 5
List of IVT reaction components

| Item | Storage Temperature |
|---|---|
| Nuclease free water | Ambient |
| Tris-HCl pH 7.0 (1M) | Ambient |
| Magnesium chloride (2M) | Ambient |
| Adenosine-5′-triphosphate (100 mM) | −20° C. |
| Cytosine-5′-triphosphate (100 mM) | −20° C. |
| Guanosine-5′-triphosphate (100 mM) | −20° C. |
| N1-methylpseudouridine | −20° C. |
| DL-dithiothreitol (DTT) (1M) | −20° C. |
| Spermidine (100 mM) | −20° C. |
| Pyrophosphatase, inorganic (100 U/mL) | −20° C. |
| RNase inhibitor, human (40,000 U/mL) | −20° C. |
| T7 RNA polymerase (50,000 U/mL) | −20° C. |
| Linear DNA template (1 mg/mL) | −20° C. |

### Example 8: Experimental Data Showing Selection of Reagent Conditions for IVT of Non-Replicating RNA

A design of experiment was designed with factors, where different levels of MgCl₂, rNTP, and CLEANCAP (C₃₂H₄₃N₁₅O₂₄P₄ (free acid)) per factor were tested (Table 6) for non-replicating RNA. A minimum number of reactions were performed to screen for effects of individual factors and their interactions, including yield (mg/mL), integrity (% full length), and dsRNA (% by mass).

TABLE 6
Experimental Data showing reagent selection for IVT of non-replicating RNA

| | MgCl₂ | rNTP | CLEANCAP | | | | MgCl₂ (Mg²⁺) |
|---|---|---|---|---|---|---|---|
| Sample | (Mg²⁺) | species | (C₃₂H₄₃N₁₅O₂₄P₄ (free | Yield | Integrity (% | dsRNA (% | to NTP |
| number | (mM) | (mM) | acid)) (AG (mM) | (mg/mL) | full length) | by mass) | species ratio |
| 1 | 16 | 8 | 8 | 3.07 | 82.8 | 1.4 | 2 |
| 2 | 16 | 8 | 2 | 3.35 | 82.5 | 1.48 | 2 |
| 3 | 16 | 8 | 5 | 3.34 | 83.1 | 1.7 | 2 |
| 4 | 16 | 2 | 5 | 2.65 | 76.9 | 3.61 | 8 |
| 5 | 16 | 5 | 5 | 5.83 | 84.6 | 3.53 | 3.2 |
| 6 | 16 | 5 | 8 | 6.11 | 86.1 | 3.38 | 3.2 |
| 7 | 16 | 5 | 2 | 5.82 | 84.8 | 3.83 | 3.2 |
| 8 | 16 | 2 | 8 | 2.59 | 74.9 | 3.54 | 8 |
| 9 | 16 | 2 | 2 | 2.7 | 51.8 | 4.15 | 8 |
| 10 | 32 | 5 | 8 | 6.34 | 80.1 | 2.83 | 6.4 |
| 11 | 32 | 2 | 2 | 2.81 | 55.2 | 2.39 | 16 |
| 12 | 32 | 5 | 2 | 5.86 | 78.6 | 2.69 | 6.4 |
| 13 | 32 | 8 | 8 | 8.96 | 84.5 | 2.66 | 4 |
| 14 | 32 | 5 | 5 | 6.12 | 79 | 2.71 | 6.4 |
| 15 | 32 | 5 | 5 | 6.25 | 79.9 | 2.74 | 6.4 |
| 16 | 32 | 8 | 2 | 8.46 | 84 | 2.42 | 4 |
| 17 | 32 | 5 | 5 | 5.84 | 80.1 | 2.62 | 6.4 |
| 18 | 32 | 8 | 5 | 8.52 | 83.6 | 2.27 | 4 |
| 19 | 32 | 2 | 8 | 2.62 | 69.9 | 2.26 | 16 |
| 20 | 32 | 5 | 5 | 5.92 | 81.7 | 2.5 | 6.4 |
| 21 | 32 | 5 | 5 | 5.94 | 80.3 | 2.4 | 6.4 |
| 22 | 48 | 5 | 5 | 5.7 | 80.3 | 1.83 | 9.6 |
| 23 | 48 | 8 | 5 | 8.52 | 82.8 | 1.71 | 6 |
| 24 | 48 | 5 | 2 | 5.35 | 80 | 1.73 | 9.6 |
| 25 | 48 | 2 | 5 | 2.58 | 65.3 | 1.53 | 24 |
| 26 | 48 | 8 | 8 | 8.64 | 80.4 | 1.68 | 6 |
| 27 | 48 | 2 | 8 | 2.66 | 67.4 | 1.48 | 24 |
| 28 | 48 | 5 | 8 | 5.53 | 80.3 | 1.77 | 9.6 |
| 29 | 48 | 8 | 2 | 8.38 | 79.1 | 2.44 | 6 |
| 30 | 48 | 2 | 2 | 2.42 | 68.2 | 1.56 | 24 |

### Example 9: Experimental Data Showing Selection of Temperature and Time for IVT of Non-Replicating RNA

A design of experiment was designed with factors, where different temperature and time were tested (Table 7) for non-replicating RNA. A minimum number of reactions was performed to screen for effects of individual factors and their interactions, including yield (mg/mL), integrity (% full length), and dsRNA (% by mass).

TABLE 7
Experimental Data showing time versus temperature

| Temperature | Time | Yield | Integrity (% | dsRNA (% |
|---|---|---|---|---|
| (C.) | (h) | (mg/mL) | full length) | by mass) |
| 37 | 4 | 5.81 | 80.3 | 2.3 |
| 33.5 | 4 | 3.81 | 84.8 | 2.3 |
| 37 | 4 | 5.77 | 81.1 | 2.23 |
| 30 | 2 | 3.04 | 84.4 | 2.76 |
| 30 | 6 | 4.33 | 80.6 | 3.03 |
| 33.5 | 4 | 4.49 | 80.7 | 2.61 |
| 30 | 2 | 2.87 | 88.6 | 3.04 |
| 30 | 4 | 2.97 | 69.3 | 2.18 |
| 33.5 | 6 | 3.87 | 63.6 | 2.25 |
| 33.5 | 6 | 4.88 | 75.4 | 2.78 |
| 37 | 2 | 4.1 | 83.2 | 1.9 |
| 37 | 6 | 6.33 | 74 | 2.41 |
| 33.5 | 2 | 4.87 | 81.7 | 2.86 |
| 33.5 | 4 | 5.47 | 72 | 2.72 |
| 37 | 4 | 5.24 | 79.9 | 2.14 |
| 33.5 | 2 | 4.34 | 68.3 | 2.49 |
| 33.5 | 4 | 4.17 | 76.5 | 2.3 |
| 30 | 4 | 4.74 | 75.3 | 2.8 |
| 30 | 6 | 5.47 | 70.6 | 2.87 |
| 37 | 6 | 5.64 | 72 | 2.07 |

### Example 10: Experimental Data Showing Selection of Reagent Conditions for IVT of Non-Replicating RNA

A design of experiment was designed with factors, where different levels of MgCl₂, rNTP, and T7 RNA polymerase concentration were tested (Table 8) for non-replicating RNA. A minimum number of reactions was performed to screen for effects of individual factors and their interactions, including yield (mg/mL), and integrity (% full length).

TABLE 8
IVT feeding/Feeding done 2 h into IVT.
Expressed as volume (fed into 100 uL IVT reaction)

| T7 RNA | | | | |
|---|---|---|---|---|
| polymerase | | MgCl₂ | | |
| (μL) | rNTP | (Mg²⁺) | | Integrity |
| (stock | (μL) | (μL) | Yield | (% full |
| concentration) | (25 mM) | (0.2 mM) | (mg/mL) | length) |
| 0 | 5 | 0 | 6.3 | 97.21 |
| 0 | 0 | 8 | 5.38 | 84.28 |
| 10 | 0 | 0 | 4.97 | 89.62 |
| 0 | 0 | 8 | 5.12 | 83.58 |
| 0 | 0 | 0 | 5.09 | 91.26 |
| 5 | 2.5 | 8 | 6.86 | 84.97 |
| 5 | 2.5 | 0 | 6.29 | 93.15 |
| 10 | 0 | 8 | 5.3 | 83.04 |
| 10 | 5 | 8 | 9.24 | 89.51 |
| 10 | 0 | 0 | 5.66 | 89.71 |
| 10 | 2.5 | 4 | 7.18 | 84.99 |
| 0 | 5 | 0 | 5.18 | 90.87 |
| 10 | 5 | 8 | 8.32 | 88.89 |
| 5 | 5 | 4 | 6.37 | 88.71 |
| 10 | 0 | 8 | 3.53 | 79.49 |
| 0 | 5 | 8 | 8.66 | 91.34 |
| 5 | 0 | 0 | 3.27 | 85.17 |
| 10 | 5 | 0 | 8.32 | 92.31 |
| 5 | 0 | 4 | 4.62 | 86.09 |
| 10 | 5 | 0 | 7.68 | 94.12 |
| 0 | 5 | 8 | 8.26 | 86.96 |
| 10 | 2.5 | 0 | 6.6 | 88.54 |
| 0 | 0 | 0 | 5.04 | 86.30 |
| 0 | 2.5 | 4 | 7.05 | 89.31 |

### Example 11: Experimental Data from Selection NTP/Mg²⁺ Ratio for IVT of Non-Replicating RNA

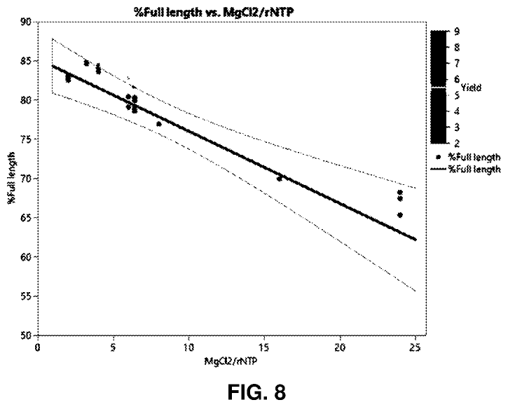

A design of experiment was designed with different levels of MgCl₂ (Mg²⁺) and rNTP, concentration were tested (FIG. 8) for non-replicating RNA. MgCl₂/NTP ratio plays a crucial role in the integrity and the yield of the IVT reaction. The more optimal results occur at lower MgCl₂/NTP ratios (3:1-8:1). Lower CLEANCAP (C₃₂H₄₃N₁₅O₂₄P₄ (free acid)) AG concentrations also play a role in the integrity and yield of the IVT reaction.
1) Kern J A, Davis R H. Application of a fed-batch system to produce RNA by in vitro transcription. Biotechnol Prog. 1999 March-April; 15(2):174-84. doi: 10.1021/bp990008g. PMID: 10194392
2) Kern, J. A. and Davis, R. H. (1997), Application of Solution Equilibrium Analysis to in vitro RNA Transcription. Biotechnol Progress, 13: 747-756. https://doi.org/10.1021/bp970094p
3) Li Zhou, Jinsong Ren, Xiaogang Qu, Nucleic acid-templated functional nanocomposites for biomedical applications, Materials Today, Volume 20, Issue 4, 2017, Pages 179-190, ISSN 1369-7021
4) Beckert B, Masquida B. Synthesis of RNA by in vitro transcription. Methods Mol Biol. 2011; 703:29-41. doi: 10.1007/978-1-59745-248-9_3. PMID: 21125481

1-66. (canceled)

67. A method for manufacturing a ribonucleic acid (RNA) by in vitro transcription (IVT), said method comprising the steps of
(A) combining in a reaction vessel components comprising:
(a) a deoxyribonucleic acid (DNA) template;
(b) a RNA polymerase; and
(c) a suitable reaction buffer, the buffer comprising
(i) Mg²⁺ at a concentration of 30-34 mM;
(ii) total NTPs at a concentration of 34-38 mM; and
(B) manufacturing the RNA.

68. The method of claim 67 wherein each species of NTP is at the same concentration.

69. The method of claim 68 wherein each species of NTP is at a concentration of 8-10 mM.

70. The method claim 67 wherein for a portion of the duration of the step of manufacturing, the temperature is 25-35° C.

71. The method of claim 70 wherein the portion is the entire step of manufacturing.

72. The method of claim 67 wherein the RNA is self-amplifying mRNA.

73. A method for manufacturing RNA by in vitro transcription (IVT), said method comprising the steps of
(A) combining in a reaction vessel components comprising:
(a) a DNA template;
(b) a RNA polymerase; and
(c) a suitable reaction buffer, the buffer comprising
(i) Mg²⁺ at a total concentration 8-20 mM;
(ii) NTPs at a concentration 12-28 mM; and
(B) manufacturing the RNA.

74. The method of claim 34, wherein the Mg²⁺ concentration in step (A) is 10-20 mM and the total NTPs concentration is 12-24 mM.

75. The method of claim 74 wherein each species of NTP is at the same concentration.

76. The method of claim 75 wherein each species of NTP is at a concentration of 2-8 mM.

77. The method of claim 73 wherein the Mg²⁺:NTP ratio is 3:1 to 8:1.

78. The method of claim 73 further comprising CLEANCAP (C32H43N15024P4 (free acid)) AG.

79. The method of claim 78 wherein the CLEANCAP AG concentration 2-10 mM.

80. The method of claim 79 wherein the CLEANCAP AG concentration is 8 mM.

81. The method of claim 73 wherein for a portion of the duration of step (A) of manufacturing, the temperature is 35-40° C.

82. The method of claim 73 wherein the mRNA is non-replicating.

83. A composition comprising:
(a) Mg²⁺ at a concentration of 10-20 mM;
(b) NTPs at a total concentration of 12-28 mM,
wherein each species of NTP is at a concentration of 2-8 mM,
said composition further comprising CLEANCAP (C32H43N15O24P4 (free acid)) AG at a concentration of 2-10 mM.
